(19) United States
(12) Patent Application Publication
(10) Pub. No.: US 2026/0096584 A1
(43) Pub. Date: Apr. 9, 2026

(54) POWDER COMPOSITION, COMBINED FOOD FOR MASKING BITTERNESS OF DRUG, METHOD FOR MASKING BITTERNESS OF DRUG, AND USE AND USE METHOD OF POWDER COMPOSITION

(71) Applicants: KRACIE, LTD., Tokyo (JP); NATIONAL CENTER FOR CHILD HEALTH AND DEVELOPMENT, Tokyo (JP)

(72) Inventors: Yuta TAKIGAWA, Osaka (JP); Tatsuya YOSHIDA, Osaka (JP); Kyoko FUKAYA, Osaka (JP); Tadashi KOBAYASHI, Osaka (JP); Akimasa YAMATANI, Tokyo (JP); Jumpei SAITO, Tokyo (JP); Sayaka KASAHARA, Tokyo (JP); Makoto KOMURA, Tokyo (JP)

(73) Assignees: KRACIE, LTD., Tokyo (JP); NATIONAL CENTER FOR CHILD HEALTH AND DEVELOPMENT, Tokyo (JP)

(21) Appl. No.: 19/112,428

(22) PCT Filed: Sep. 13, 2023
(86) PCT No.: PCT/JP2023/033361

§ 371 Date: Mar. 17, 2025

(30) Foreign Application Priority Data
Sep. 20, 2022 (JP) 2022-148830

(51) Int. Cl. A23L 27/00; A23L 27/30; A23P 30/40; A61K 9/00; A61K 31/167; A61K 31/4164; A61K 31/573; A61K 31/635; B65D 6/02

(52) U.S. Cl. CPC A23L 27/84; A23L 27/30; A23P 30/40; A61K 9/0053; A61K 31/167; A61K 31/4164; A61K 31/573; A61K 31/635; B65D 7/02

(57) ABSTRACT

Provided is a powder composition that can be made into a foamy food product exhibiting a masking effect and being highly dispersible in an aqueous solvent. The powder composition is to be made into a foamy food product by mixing with water. The powder composition comprises the following components (A) to (D): (A) a carbohydrate; (B) an acid component; (C) a carbonate component; and (D) a foam stabilizer.

### TECHNICAL FIELD

The present invention relates to a powder composition that can be used to produce a foamy food product that exhibits a masking effect and has excellent dispersibility in an aqueous solvent. More specifically, the present invention relates to a powder composition that can mask bitterness of drug or the like and ensure the dissolution of the drug when the drug or the like incorporated into the foamy food product according to the powder composition of the present invention is ingested, and the combined food for masking bitterness of the drug and the method for masking bitterness of the drug.

### BACKGROUND ART

The present inventors have proposed a combined confectionery (see, for example, PTL 1) and a powder composition (see, for example, PTL 2) as powder compositions for preparing foods that foam and expand when mixed with water. The combined confectionery allows the user to enjoy the change in physical properties and appearance of the powder composition for paste, which foams more the more it is kneaded by contacting it with water, causing the paste-like product to expand and become cloudy. The powder composition also allows the user to enjoy the visual change of the powder composition, which foams and expands instantly when a liquid component is added. That is, in both cases, a foamy food is prepared while enjoying the process of mixing with water, in which the powder composition foams and expands, and the foamy food is eaten as it is. However, the invention does not disclose the inclusion of a drug in the foamy food and the taking of the drug, or the masking of the bitterness of the drug.

On the other hand, a foamy composition comprising a polymeric foam stabilizer, a non-cellulosic polysaccharide, and a pharmaceutical active ingredient for masking the bitter or unpleasant taste of a pharmaceutical active agent or dietary supplement is known (see, for example, PTL 3). The foamy composition is prepared by vigorously stirring a foam generating device such as a mixer or a former to create a foam, and then drying it. In addition, a coated pharmaceutical is used to protect the pharmaceutical active ingredient during the preparation of the foam or foamy composition. Furthermore, when a pharmaceutical active ingredient is added after preparing a foamy composition first, for example, nicotine is applied to the surface of the foamy composition after drying using a solvent such as acetone. Therefore, regardless of the method, there is a problem in terms of convenience when the consumer uses it to mask bitter pharmaceuticals and the like.

In addition, when taking bitter drugs and the like, known medication-assisting food used to mask the bitterness include bitter-masking granular jelly drinks (see, for example, PTL 4), powder compositions that are made into gel foods by adding water (see, for example, PTL 5), and medication-assisting agent made of chocolate with a viscosity in the range of 50 to 500 poises in the temperature range of 10 to 40° C. (see, for example, PTL 6).

However, when the drug is taken using the granular jelly drink or the powder composition, the drug may taste bitter and a stable masking effect may not be obtained. In addition, since the medication aid using chocolate has poor dispersibility in aqueous solvents, it is difficult to measure the dissolution of the drug by a dissolution test according to the Japanese Pharmacopoeia.

### CITATION LIST

### Patent Literature

PTL 1: JP H03-33274 Y2
PTL 2: JP H06-237696 A
PTL 3: JP 2000-325028 A
PTL 4: WO 2005/025622 A
PTL 5: JP 2012-105574 A
PTL 6: JP 2017-48169 A

### SUMMARY OF INVENTION

### Technical Problem

The present invention has been made in consideration of the above circumstances, and its object is to provide a powder composition that can be used to form a foamy food product that has a masking effect and excellent dispersibility in aqueous solvents.

More specifically, the object of the present invention is to provide a powder composition that can mask bitterness of drug and ensure the dissolution of the drug when the drug incorporated into the foamy food product according to the powder composition of the present invention is ingested, a combined food for masking bitterness of drug, and a method for masking bitterness of drug.

### Solution to Problem

The present inventors have paid attention to foamy food products as a candidate for a medication-assisting food that exerts a masking effect and has excellent dispersibility in aqueous solvents, and have conducted intensive research. As a result, they have found that by adding water to a powder composition comprising carbohydrate, acid component, carbonate component, and foam stabilizer to make foamy food products, it is possible to envelope and take drug, mask bitterness of drug, and provide good dispersibility in aqueous solvents, and it is possible to mask bitterness of drug, especially the bitterness immediately after taking it, and also ensure the dissolution of drug, and have arrived at the present invention.

The first invention of the present application is a powder composition for mixing with water to form a foamy food product, comprises the following components (A) to (D):
(A) a carbohydrate,
(B) an acid component,
(C) a carbonate component, and
(D) a foam stabilizer.

The powder composition according to the first invention of the present application preferably further comprises the following component (E):
(E) Masking component.

In the powder composition according to the first invention of the present application, the specific gravity of the foamy food product is more preferably 0.83 g/cc or less.

In the powder composition according to the first invention of the present application, the foamy food product is more preferably for enveloping a drug and taking it.

The second invention of the present application is a combined food for masking bitterness of drug, comprising the above powder composition and (X) a container having a two-tiered recess in the shape of an inverted Kagami-mochi (rice cake).

The third invention of the present application is a method for masking bitterness of drug, which comprises the steps of: adding water to and mixing a powder composition comprising a carbohydrate, an acid component, a carbonate component, and a foam stabilizer to prepare a foamy food product, and mixing the drug into the foamy food product.

The fourth invention of the present application relates to a use of a powder composition for adding water to and mixing the powder composition to obtain a foamy food product; enveloping a drug in the foamy food product; and taking it, wherein the powder composition comprises the following components (A) to (D):
(A) a carbohydrate,
(B) an acid component,
(C) a carbonate component, and
(D) a foam stabilizer.

The fifth invention of the present application is a method for using a powder composition, which comprises the steps of: adding water to and mixing a powder composition to obtain a foamy food product, and enveloping a drug in the foamy food product and taking it, wherein the powder composition comprises the following components (A) to (D):
(A) a carbohydrate,
(B) an acid component,
(C) a carbonate component, and
(D) a foam stabilizer.

### Advantageous Effects of Invention

The foamy food product obtained by adding water to the powder composition of the present invention has a stable masking effect. Furthermore, by preparing a foamy food product by adding water to the powder composition of the present invention, a foamy food product having a masking effect superior to that of conventional medication-assisting food can be obtained.

Since the foamy food product prepared using the powder composition of the present invention has excellent dispersibility in aqueous solvents, even if a drug is contained in the foam food product, a dissolution test can be carried out using a method in accordance with the Japanese Pharmacopoeia.

### BRIEF DESCRIPTION OF THE DRAWINGS

FIG. 1 is a schematic diagram showing how bitterness of drug is masked.

FIGS. 2A and 2B are diagrams showing an example of a container provided for the combined food according to the present embodiment.

FIG. 3 is a graph showing the results of a dissolution test of acetaminophen according to the dissolution test of the example.

FIG. 4 is a graph showing the results of a dissolution test of prednisolone according to the dissolution test of the example.

FIG. 5 is a diagram showing the results of a dissolution test of furosemide according to the dissolution test of the example.

FIG. 6 is a graph showing the results of a dissolution test of metronidazole according to the dissolution test of the example.

FIG. 7 shows the results of a questionnaire asking about prior purchase intentions for the combined food of the present invention.

FIG. 8A is a questionnaire result showing the masking effect of the combined food of the present invention to consumers, and FIG. 8B is a questionnaire result showing the masking effect of the reference food to consumers.

FIG. 9A is a result of a questionnaire showing the ease of eating of the bittering agent enveloped in the combined food of the present invention, and FIG. 9B is a result of a questionnaire showing the ease of eating of the bittering agent enveloped in the reference example.

FIG. 10A shows the results of a questionnaire survey showing the masking effect felt by parents of the combined food of the present invention, and FIG. 10B shows the results of a questionnaire survey showing the masking effect felt by parents of the reference example.

FIG. 11A shows the results of a questionnaire survey showing the ease of eating felt by parents of the bittering agent enveloped in the combined food of the present invention, and FIG. 11B shows the results of a questionnaire survey showing the ease of eating felt by parents of the bittering agent enveloped in the reference example.

FIG. 12 shows the results of a questionnaire asking about purchasing intentions after use of the combined food of the present invention.

### DESCRIPTION OF EMBODIMENTS

The present invention will be described in detail below.

### (Regarding Powder Compositions)

It is important that the powder composition of the present invention comprises (A) a carbohydrate, (B) an acid component, (C) a carbonate component, and (D) a foam stabilizer in order to prepare a foamy food product that exhibits a stable masking effect.

In this specification, “masking effect” means the effect of reducing bitterness that occurs when a bitter substance binds to bitterness receptors on the tongue, and “stable masking effect” means that the degree of bitterness reduction is reproducible when multiple people take the drug multiple times.

In this specification, “including a drug” means that the drug is envelop in the foamy food product, and the purpose is to reduce the surface exposure of the drug by mixing or enveloping the drug in the foamy food product.

In this specification, “dissolution of a drug” means the rate at which the active ingredient in the drug dissolves into a test liquid in a dissolution test. In the present examples, it means the rate at which the active ingredient in the drug mixed or enveloped in the foamy food product dissolves into the test liquid.

### <(A) Carbohydrate>

The component (A) used in the present invention is a carbohydrate. In the present invention, the carbohydrate is used as a component that is the base material of the foamy food product. The carbohydrate is not particularly limited as long as it is a powdered sweetener. Specific examples of carbohydrates include glucose, fructose, sugar, maltose, lactose, oligosaccharides, dextrin, powdered starch syrup, sugar alcohol and the like. Appropriately selected from these, they may be used alone or in combination of two or more. Among these, sugar is preferably used because it can exert an excellent masking effect and can give the foamy food product a good flavor.

The sugar content is preferably 85.0% by weight or more based on the total weight of the powder composition. If the sugar content is 85.0% by weight or more, a stable and high masking effect can be obtained from immediately after ingestion until swallowing.

### <(B) Acid Component>

The component (B) used in the present invention is an acid component. In the present invention, the acid component acts as a proton donor to carbonate ions and is used for the purpose of producing carbon dioxide. Examples of the acid component include organic acids such as citric acid, tartaric acid, malic acid, and ascorbic acid, and may be appropriately selected and used alone or in combination of two or more. Among these, citric acid, tartaric acid, and malic acid are preferably used in that they can improve the masking effect.

The acid content is preferably 0.4% by weight or more based on the total weight of the powder composition. If the acid content is 0.4% by weight or more, sufficient bubbles (carbon dioxide) necessary for masking can be obtained, and the flavor of the foamy food product can be improved.

### <(C) Carbonate Component>

The component (C) used in the present invention is a carbonate component. The carbonate component is a carbonate salt that dissolves in water and releases carbonate ions. In the present invention, the carbonate component is used for the purpose of producing carbonate ions, which are carbon dioxide precursors. Examples of the carbonate component include sodium bicarbonate and calcium carbonate, and they may be appropriately selected and used alone or in combination of two or more. Among these, sodium bicarbonate is preferably used because it can improve the masking effect and can improve the flavor of the foamy food product.

The carbon dioxide content is preferably 0.4% by weight or more based on the total weight of the powder composition. If the content is 0.4% by weight or more, a sufficient amount of air bubbles (carbon dioxide) required for masking can be obtained.

The weight ratio of the acid component (B) to the carbonate component (C) is preferably 1:0.25 to 4 (see, for example, Examples 20-23). This allows efficient and stable generation of bubbles (carbon dioxide), and makes it possible to prepare a foamy food product that has a high masking effect. The weight ratio of the acid component (B) to the carbonate component (C) is more preferably 1:1. This allows the preparation of a foamy food product with a low specific gravity.

### <(D) Foam Stabilizer>

The component (D) used in the present invention is a foam stabilizer. In the present invention, the foam stabilizer is used for the purpose of stabilizing the foamy food product, that is, stably maintaining the structure of the foamy food product including a drug. Examples of foam stabilizers include egg white, soy protein, Quillaja saponin, starch sodium octenyl succinate and the like, and may be appropriately selected and used alone or in combination of two or more. Among these, the combined use of Quillaja saponin and starch sodium octenyl succinate is preferably used in that it can maintain the structure of the foamy food product from immediately after taking until swallowing, and can obtain a sufficient masking effect, as well as improve the flavor of the foamy food product.

The content of the foam stabilizer is preferably 1.3% by weight or more based on the total weight of the powder composition. If the content is 1.3% by weight or more, a sufficient masking effect can be obtained from immediately after ingestion until swallowing.

In addition, it is important to add water to the powder composition comprising the components (A) to (D) of the present invention to prepare a foamy food product, in order to provide a stable masking effect. Here, “adding water and mixing” means adding water at room temperature (10 to 30° C.) to the powder composition and stirring and mixing. Furthermore, “foamy food product” means a non-dried food containing air bubbles. It is preferable that the foamy food product is prepared just before use when taking drug and the like.

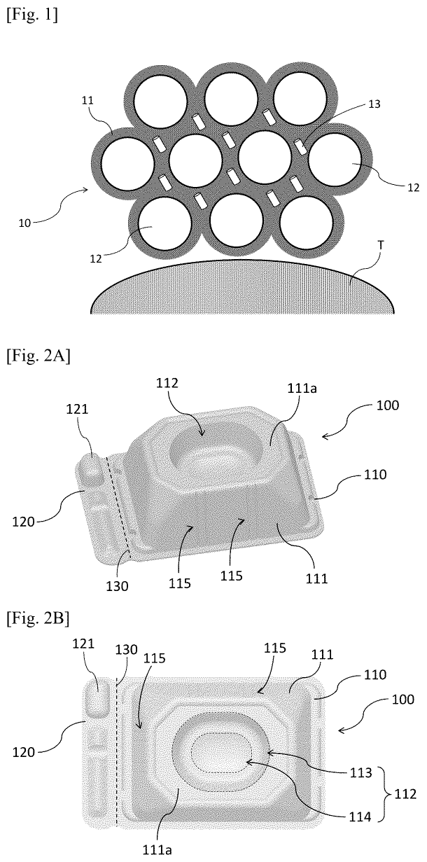

It is believed that a foamy food product prepared using the powder composition comprising the components (A) to (D) of the present invention can reduce the chance of the active ingredient (bitter ingredient) in a drug coming into direct contact with the tongue when mixed with the drug. This will be explained with reference to FIG. 1.

FIG. 1 is a schematic diagram showing how bitterness of drug is masked, in which 10 is a drug-including foamy food product, 11 is a foamy food product, 12 is air bubbles, and 13 is a drug. T represents a human tongue, and FIG. 1 represents the inside of a human mouth. In FIG. 1, the foamy food product 11 has a foam structure in which a plurality of air bubbles 12 are gathered. As shown in FIG. 1, in the drug-including foamy food product 10, the drug 13 containing a bitter ingredient is in a state of being enveloped in the foam structure of the foamy food product 11 prepared using the powder composition according to the present invention. As a result, when taken orally, the foamy food product 11 according to the present invention exhibits the following actions (1) to (3), which reduces the opportunity for the active ingredient (bitter ingredient) in the drug 13 to come into direct contact with the tongue, and it is presumed that a good masking effect can be obtained.
(1) Suppression of surface exposure of the drug 13 by being enveloped in the foam structure of the foamy food product 11
(2) Suppression of diffusion of the drug 13 in the mouth due to the high shape retention of the foamy food product 11
(3) Suppression of dissolution of the drug 13 in water by due to the low water content of the foamy food product 11.

### <(E) Masking Component>

The component (E) used in the present invention is a masking component. In the present invention, the masking component is suitable because it shows a higher masking effect not only immediately after taking the component, but also during chewing and after swallowing. Examples of the masking component include sugar cane extract, sucralose, thaumatin, erythritol and the like, and may be appropriately selected and used alone or in combination of two or more kinds. Among these, sugar cane extract is preferably used because it can show a better masking effect and can improve the flavor of foamy food product.

The content of the masking component is preferably 0.7% by weight or more of the total weight of the powder composition. If the content is 0.7% by weight or more, the component (E) is added to the base material of the component (A), and a higher masking effect can be obtained not only immediately after administration, but also during chewing and after swallowing.

In addition to the above ingredients, auxiliary raw materials may be added as long as the purpose of the present invention is not impaired. Examples of auxiliary raw materials include starch, thickening polysaccharides, emulsifiers, flavorings, colorings and the like. These may be appropriately selected and used alone or in combination of two or more.

The powder composition of the present invention is produced, for example, as follows: First, (A) a carbohydrate, (B) an acid component, (C) a carbonate component, (D) a foam stabilizer, and, if necessary, (E) a masking component and/or auxiliary raw materials are prepared, and these are powder-mixed to obtain the powder composition of the present invention.

### (Regarding Foamy Food Product)

The powder composition of the present invention is preferably used as a foamy food product for enveloping a drug when the drug is orally taken. The foamy food product obtained by using the powder composition of the present invention will be described below. For example, the foamy food product is prepared from the powder composition as follows.

First, 0.28 to 0.36 parts by weight of water at room temperature is added to 1 part by weight of the powder composition, and the mixture is stirred and mixed with a stirring tool such as a spoon at a speed of about 2 rotations per second for about 30 rotations to obtain a foamy food product. The specific gravity of the foamy food product at this time is preferably 0.83 g/cc or less. This makes it possible to obtain a foamy food product that exhibits a higher masking effect. More preferably, the pH of the foamy food product at this time is designed to be 5.0 to 7.0, which is suitable in terms of preventing the dissolution of drugs (such as antibiotics) that dissolve under acidic conditions.

Then, a minimum dose of a drug or the like is mixed and wrapped around 5.6 to 6.9 g of the foamy food product, for example, using a small spoon such as a teaspoon, in a concentric pattern at a speed of about 2 rotations per second for about 10 rotations, so as not to destroy the bubbles in the foamy food product as much as possible. The drug-including foamy food product thus prepared is orally taken by the subject of drug intake. In addition, the term “including” of the drug-including foamy food product means that the drug is envelop in the foamy food product. The purpose is to reduce the surface exposure of the drug by mixing or wrapping the drug in the foamy food product.

The above-mentioned drugs include drugs taken orally. The forms of the drugs include tablets, capsules, granules, powders, oral liquids, syrups, oral jellies, oral films, and oral tablets. Examples of tablets include prednisolone tablets (e.g., “PREDONINE (registered trademark) tablets 5 mg”) and metronidazole tablets (e.g., “FLAGYL (registered trademark) oral tablets 250 mg”), and examples of granules include acetaminophen fine granules (e.g., “CALONAL (registered trademark) fine granules 20%”, “CALONAL (registered trademark) fine granules 50%” and the like), and furosemide fine granules (e.g., “Furosemide fine granules 4% “EMEC”” and the like).

In addition, when preparing the foamy food product, the container used for adding water and mixing may be, for example, a suitable dish such as a plate or a bowl. In addition, (X) a container having a two-tiered recess in the shape of an inverted Kagami-mochi may be prepared in advance, and the powder composition may be packaged together with the container to form a combined food. This combined food is an example of the combined food for masking bitterness of drug of the present invention.

The combined food according to this embodiment includes the powder composition according to the embodiment of the present invention described above and a container (X) having a two-tiered recess in the shape of an inverted Kagami-mochi (two-tiered round rice cake). FIG. 2A and FIG. 2B show the appearance of one example of the container (X) (container 100).

FIG. 2A is a perspective view of the container 100. FIG. 2B is a plan view of the container 100. The container 100 is formed of, for example, a relatively lightweight plastic. The container 100 is composed of a main body 110 and a spoon part 120. A perforation 130 is provided between the main body 110 and the spoon part 120. When the container 100 is used, the spoon part 120 is separated from the main body 110.

The main body 110 has a raised part 111 that rises in a generally truncated pyramid shape. A two-tiered recess 112 having an inverted Kagami mochi shape is provided on a top surface 111a of this raised part 111. When preparing a foamy food product using the combination food of this embodiment, the powder composition and water that are the ingredients of the foamy food product, as well as a drug, are placed into the two-tiered recess 112. The volume of the two-tiered recess 112 can be appropriately determined taking into consideration the amount of the powder composition contained in the combination food, the amount of water to be added, and the volume of the foamy food product to be formed.

The two-tiered recess 112 has a first recess 113 and a second recess 114 (see FIG. 2B). The second recess 114 is formed by further hollowing out the bottom surface of the first recess 113. The depth of the second recess 114 from the bottom surface of the first recess 113 can be, for example, 1 mm or more and 5 mm or less. In addition, if the depth of the second recess 114 is determined taking into consideration the volume of water added when preparing the foamy food product, it is preferable in that the foamy food product can be prepared without a measuring spoon, which will be described later. Furthermore, it is preferable to form a plurality of grooves 115 on the side surface of the raised part 111 as shown in FIG. 2, in that the strength of the main body 110 can be maintained.

The spoon part 120 is used to stir the ingredients put into the two-tiered recess 112 when preparing a foamy food product. The spoon part 120 is formed with a third recess 121. The capacity of the third recess 121 is preferably determined taking into consideration the amount of water put in when preparing a foamy food product. This allows the spoon part 120 to be used as a measuring spoon.

As described above, the combined food according to the present embodiment includes a powder composition and a container 100 having an inverted Kagami-mochi-shaped two-tiered recess 112. This allows foamy food products and drug-including foamy food products to be easily prepared using the container 100 included in the combined food without the need for a separate container or the like.

In another embodiment, the container (X) may be formed only by the main body 110. In this case, it is preferable that the combined food includes a stirring spoon or the like as an accessory separate from the main body 110.

### (Regarding Method for Masking Bitterness of Drugs)

The present invention includes a method for masking bitterness of drug by adding water to and mixing a powder composition comprising a carbohydrate, an acid component, a carbonate component, and a foam stabilizer to prepare a foamy food product, and then mixing the drug into the resulting foamy food product.

Examples of the powder composition used in this method include the powder composition described above. Examples of the foamy food product obtained by adding water to and mixing the powder composition include the foamy food product described above. Examples of the drug to be mixed into the foamy food product include the drugs described above. According to this method, when a drug containing an active ingredient (i.e., a bitter ingredient) is orally taken, the opportunity for the bitter ingredient to come into direct contact with the tongue can be reduced, thereby reducing the bitterness felt by the taker and reducing the discomfort felt when taking the drug.

### (Regarding Use of the Powder Composition)

The present invention includes the use of a powder composition. The use is for taking a drug enveloped in the foamy food product which is formed by mixing the powder composition with water.

Examples of the powder composition used in the use include the powder composition described above. Examples of foamy food products obtained by adding water to and mixing the powder composition include the foamy food product described above. Examples of drugs to be mixed into the foamy food product include the drugs described above. The use of this powder composition can reduce the chance of the bitter ingredient directly touching the tongue when a drug containing an active ingredient (i.e., a bitter ingredient) is orally ingested, thereby reducing the bitterness felt by the taker and reducing the discomfort felt when taking the drug.

### (Regarding Method for Using the Powder Composition)

The present invention includes a method for using the powder composition, which comprises adding water to and mixing the powder composition to obtain a foamy food product, and enveloping a drug in the foamy food product and taking the drug.

Examples of the powder composition used in the use method include the powder composition described above. Examples of foamy food products obtained by adding water to and mixing the powder composition include the foamy food products described above. Examples of drugs to be mixed into the foamy food products include the drugs described above. According to this use method, when a drug containing an active ingredient (i.e., a bitter ingredient) is orally ingested, the opportunity for the bitter ingredient to come into direct contact with the tongue can be reduced, thereby reducing the bitterness felt by the taker and reducing the discomfort felt when taking the drug.

### EXAMPLE

The present invention will be described in detail below with reference to examples of the present invention, but the present invention is not limited to these.

### [Confirmation Test of Masking Effect]

A foamy food product was prepared from the powder composition according to one embodiment of the present invention, and the bitterness-masking effect was confirmed.

### <Preparation of Powder Composition>

First, in each of the Examples and Comparative Examples, the raw materials shown in Tables 1 to 3 were mixed in powder form to prepare powder compositions having the compositions shown in the tables.

### <Preparation of Foamy Food Product>

The foamy food product was then prepared according to the following procedure.

### 1. Method for Preparing Foamy Food Products (*1 in Each Table)

(1) Water (15° C.) was added to the powder composition so as to obtain the weight ratio shown in the table.

(2) The mixture was stirred with a plastic spoon at a speed of 2 rotations per second for 30 rotations.

### <Measurement of Specific Gravity of Foamy Food Product>

The specific gravity of the resulting foamy food product was calculated by the following method.

### 2. How to Calculate Specific Gravity (*2 in Each Table)

The foamy food product was filled to the level of 30 cc container, and the weight was measured and the specific gravity was calculated.

### <Masking Evaluation>

The obtained foamy food product was mixed with a bittering agent to prepare a foamy food product containing a bittering agent, which was then taken and evaluated (*3 in each table). The evaluation was carried out by six expert panelists. The specific method is as follows.

### 3. How to Evaluate the Masking Effect

### 3-1 Masking Target

The target for masking was a bittering agent prepared as follows using a quassia extract (“DC Bitter #20023” manufactured by Takada Fragrance Co., Ltd.), which is a bittering component.

10% DC Bitter #20023, 45% corn starch, and 45% lactose were mixed and extruded to prepare a granular bittering agent.

### 3-2 Evaluation Method

(1) 0.5 g of a bittering agent was added to 6.8 g of a previously prepared foamy food product.

(2) A plastic spoon was used to stir the mixture at a speed of 2 rotations per second for 10 rotations to prepare a foamy food product containing a bittering agent.

(3) One teaspoon of foamy food product containing a bittering agent was taken.

(4) The taste was evaluated at three points: A: immediately after taking the tablet (the first taste felt when put in the mouth), B: during chewing (a taste between A and C), and C: after swallowing (the taste that lingers after swallowing).

### 3-3 Evaluation Criteria, etc.

0.5 g of the bittering agent was placed in the mouth in the same way as a normal powdered drug and washed down with water. The maximum bitterness felt when this was measured was the upper limit (5 points), and the lower limit (0 points) was the complete lack of bitterness felt.
Relative evaluation was done on a 6-point scale ranging from 5 points (maximum bitterness) to 0 points (no bitterness felt at all).
Ratings A to C are the average of six expert panelists.
The final evaluation was determined as follows based on the average of A to C.
Excellent: 0 points or more, 1.5 points or less
Good: more than 1.5 points, 2 points or less
Bad: more than 2 points, 5 points or less

### Examples 1 to 4, Comparative Examples 1 to 4

Table 1 below shows the compositions of the powder compositions of Examples 1 to 4 and Comparative Examples 1 to 4, the specific gravities of the foamy food products, and the masking evaluation results.

TABLE 1
(Parts by weight)

| | | | | | Comparative | Comparative |
|---|---|---|---|---|---|---|
| | | | Example 1 | Example 2 | Example 1 | Example 2 |
| Powder | Carbohydrates | sugar | 71.54 | 71.54 | — | 71.54 |
| Composition | | Glucose | 3.20 | 3.20 | — | 3.20 |
| | | Dextrin | 13.69 | 13.69 | — | 13.69 |
| | Acid component | Citric acid | 1.65 | 1.65 | 1.65 | — |
| | Carbonate component | Sodium bicarbonate | 1.65 | 1.65 | 1.65 | 1.65 |
| | Foam Stabilizers | Quillaja saponin | 0.43 | 0.43 | 0.43 | 0.43 |
| | | Starch Octenyl Succinate | 2.14 | 2.14 | 2.14 | 2.14 |
| | | Sodium | | | | |
| | Masking components | Sugarcane extract | — | 1.40 | 1.40 | 1.40 |
| | Auxiliary raw materials | Tapioca starch | 3.00 | 3.00 | 3.00 | 3.00 |
| | | Xanthan gum | 0.35 | 0.35 | 0.35 | 0.35 |
| | | Fragrances | 0.65 | 0.65 | 0.65 | 0.65 |
| | | Coloring | 0.30 | 0.30 | 0.30 | 0.30 |

| | | | | |
|---|---|---|---|---|
| total | 98.60 | 100.00 | 11.57 | 98.35 |
| Carbohydrate content (wt %) | 89.69 | 88.43 | 0.00 | 89.91 |
| Acid component content (wt %) | 1.67 | 1.65 | 14.26 | 0.00 |
| Carbonate component content (wt %) | 1.67 | 1.65 | 14.26 | 1.68 |
| Foam stabilizer content (wt %) | 2.61 | 2.57 | 22.21 | 2.61 |
| Masking component content (wt %) | 0.00 | 1.40 | 12.10 | 1.42 |

| Foamy food product *1 | Powder composition: water | 5.23:1.50 | 5.30:1.50 | 0.61:1.50 | 5.21:1.50 |
|---|---|---|---|---|---|
| | (weight ratio) | | | | |
| | Specific gravity (g/cc) *2 | 0.39 | 0.43 | 0.26 | 1.29 |
| Masking evaluation *3 | A: Immediately after taking | 0.50 | 0.50 | 2.83 | 1.00 |
| | B: During chewing | 1.33 | 1.00 | 3.83 | 1.83 |
| | (between A and C) | | | | |
| | C: After swallowing | 2.83 | 2.17 | 4.17 | 3.33 |
| | Average of A to C | 1.56 | 1.22 | 3.61 | 2.06 |
| | Final evaluation | Good | Excellent | Bad | Bad |

(Parts by weight)

| | | | Comparative | Comparative | | |
|---|---|---|---|---|---|---|
| | | | Example 3 | Example 4 | Example 3 | Example 4 |
| Powder | Carbohydrates | sugar | 71.54 | 71.54 | 71.54 | 71.54 |
| Composition | | Glucose | 3.20 | 3.20 | 3.20 | 3.20 |
| | | Dextrin | 13.69 | 13.69 | 13.69 | 13.69 |
| | Acid component | Citric acid | 1.65 | 1.65 | 0.83 | 0.41 |
| | Carbonate component | Sodium bicarbonate | — | 1.65 | 0.83 | 0.41 |
| | Foam Stabilizers | Quillaja saponin | 0.43 | — | 0.43 | 0.43 |
| | | Starch Octenyl Succinate | 2.14 | — | 2.14 | 2.14 |
| | | Sodium | | | | |
| | Masking components | Sugarcane extract | 1.40 | 1.40 | 1.40 | 1.40 |
| | Auxiliary raw materials | Tapioca starch | 3.00 | 3.00 | 3.00 | 3.00 |
| | | Xanthan gum | 0.35 | 0.35 | 0.35 | 0.35 |
| | | Fragrances | 0.65 | 0.65 | 0.65 | 0.65 |
| | | Coloring | 0.30 | 0.30 | 0.30 | 0.30 |

| | | | | |
|---|---|---|---|---|
| total | 98.35 | 97.43 | 98.35 | 97.53 |
| Carbohydrate content (wt %) | 89.91 | 90.76 | 89.91 | 90.67 |
| Acid component content (wt %) | 1.68 | 1.69 | 0.84 | 0.42 |
| Carbonate component content (wt %) | 0.00 | 1.69 | 0.84 | 0.42 |
| Foam stabilizer content (wt %) | 2.61 | 0.00 | 2.61 | 2.64 |
| Masking component content (wt %) | 1.42 | 1.44 | 1.42 | 1.44 |

| Foamy food product *1 | Powder composition: water | 5.21:1.50 | 5.16:1.50 | 5.21:1.50 | 5.17:1.50 |
|---|---|---|---|---|---|
| | (weight ratio) | | | | |
| | Specific gravity (g/cc) *2 | 1.34 | 0.42 | 0.53 | 0.78 |
| Masking evaluation *3 | A: Immediately after taking | 1.00 | 0.83 | 0.67 | 0.83 |
| | B: During chewing | 1.83 | 2.00 | 1.67 | 1.67 |
| | (between A and C) | | | | |
| | C: After swallowing | 3.33 | 3.33 | 2.67 | 3.17 |
| | Average of A to C | 2.06 | 2.06 | 1.67 | 1.89 |
| | Final evaluation | Bad | Bad | Good | Good |

As a result of the evaluation, each Example showed an excellent masking effect. Among them, the powder compositions of Examples 1 and 2 were able to prepare a strong foamy food product. Moreover, Example 2 showed a very good masking effect not only immediately after administration, but also during chewing and after swallowing. Moreover, the results of Examples 2, 3, and 4 confirmed that the masking effect tends to increase as the specific gravity of the foamy food product decreases. On the other hand, in Comparative Examples 1 to 4, it was not possible to prepare a foamy food product, and a sufficient masking effect was not obtained.

From the above results, it was confirmed that a foamy food product can be prepared by using a powder composition comprising a carbohydrate, an acid component, a carbonate component, and a foam stabilizer, and that the bitterness can be masked. In addition, it was confirmed that the bitterness can be effectively masked from immediately after taking the food until after swallowing by using a masking component in combination.

### Examples 5 to 24

Tables 2 and 3 below show the compositions of the powder compositions of Examples 5 to 24, the specific gravities of the foamy food products, and the masking evaluation results.

TABLE 2
(Parts by weight)

| | | | Example 5 | Example 6 | Example 7 | Example 8 | Example 9 | Example 10 |
|---|---|---|---|---|---|---|---|---|
| Powder | Carbohydrates | sugar | 71.54 | 71.54 | 71.54 | 71.54 | 71.54 | 71.54 |
| Composition | | Glucose | 3.20 | 3.20 | 3.20 | 3.20 | 3.20 | 3.20 |
| | | Dextrin | 13.69 | 13.69 | 13.69 | 13.69 | 13.69 | 13.09 |
| | Acid | Citric acid | | | | 1.65 | 1.65 | 1.65 |
| | components | Tartaric acid | 1.65 | | | | | |
| | | Malic acid | | 2.65 | | | | |
| | | Ascorbic acid | | | 1.65 | | | |
| | Carbonate | Sodium bicarbonate | 1.65 | 1.65 | 1.65 | | 1.65 | 1.65 |
| | components | Calcium carbonate | | | | 1.65 | | |
| | Foam | Quillaja saponin | 0.43 | 0.43 | 0.43 | 0.43 | 2.57 | |
| | Stabilizers | Starch Octenyl Succinate | 2.14 | 2.14 | 2.14 | 2.14 | | 2.57 |
| | | Sodium | | | | | | |
| | | Egg white | | | | | | |
| | | Soy Protein | | | | | | |
| | Masking | Sugarcane extract | 1.40 | 1.40 | 1.40 | 1.40 | 1.40 | 1.40 |
| | components | Sucralose | | | | | | |
| | | Thaumatin | | | | | | |
| | | Erythritol | | | | | | |
| | Auxiliary raw | Tapioca starch | 3.00 | 3.00 | 3.00 | 3.00 | 3.00 | 3.00 |
| | materials | Xanthan gum | 0.35 | 0.35 | 0.35 | 0.35 | 0.35 | 0.35 |
| | | Fragrances | 0.65 | 0.65 | 0.65 | 0.65 | 0.65 | 0.65 |
| | | Coloring | 0.30 | 0.30 | 0.30 | 0.30 | 0.30 | 0.30 |

| | | | | | | |
|---|---|---|---|---|---|---|
| total | 100.00 | 100.00 | 100.00 | 100.00 | 100.00 | 100.00 |
| Carbohydrate content (wt %) | 88.43 | 88.43 | 88.43 | 88.43 | 88.43 | 88.43 |
| Acid component content (wt %) | 1.65 | 1.65 | 1.65 | 1.65 | 1.65 | 1.65 |
| Carbonate component content (wt %) | 1.65 | 1.65 | 1.65 | 1.65 | 1.65 | 1.65 |
| Foam stabilizer content (wt %) | 2.57 | 2.57 | 2.57 | 2.57 | 2.57 | 2.57 |
| Mashing component content (wt %) | 1.40 | 1.40 | 1.40 | 1.40 | 1.40 | 1.40 |

| Foamy food product *1 | Powder composition: water | 5.30:1.50 | 5.30:1.50 | 5.30:1.50 | 5.30:1.50 | 5.30:1.50 | 5.30:1.50 |
|---|---|---|---|---|---|---|---|
| | (weight ratio) | | | | | | |
| | Specific gravity (g/cc) *2 | 0.36 | 0.41 | 0.83 | 0.59 | 0.36 | 0.41 |
| Masking evaluation *3 | A: Immediately after taking | 0.50 | 0.50 | 0.83 | 0.67 | 0.33 | 0.67 |
| | B: During chewing | 1.17 | 1.50 | 1.67 | $.67 | 0.83 | 1.50 |
| | (between A and C) | | | | | | |
| | C: After swallowing, | 2.33 | 2.33 | 2.83 | 2.83 | 2.50 | 2.00 |
| | Average of A to C | 1.33 | 1.44 | 1.78 | 1.72 | 1.22 | 1.39 |
| | Final evaluation | Excellent | Excellent | Good | Good | Excellent | Excellent |

(Parts by weight)

| | | | Example 11 | Example 12 | Example 13 | Example 14 | Example 15 |
|---|---|---|---|---|---|---|---|
| Powder | Carbohydrates | sugar | 71.54 | 71.54 | 71.54 | 71.54 | 71.54 |
| Composition | | Glucose | 3.20 | 3.20 | 3.20 | 3.20 | 3.20 |
| | | Dextrin | 13.69 | 13.69 | 13.69 | 13.69 | 13.69 |
| | Acid | Citric acid | 1.65 | 1.65 | 1.65 | 1.65 | 1.65 |
| | components | Tartaric acid | | | | | |
| | | Malic acid | | | | | |
| | | Ascorbic acid | | | | | |
| | Carbonate | Sodium bicarbonate | 1.65 | 1.65 | 1.65 | 1.65 | 1.65 |
| | components | Calcium carbonate | | | | | |
| | Foam | Quillaja saponin | | | 0.43 | 0.43 | 0.43 |
| | Stabilizers | Starch Octenyl Succinate | | | 2.14 | 2.14 | 2.14 |
| | | Sodium | | | | | |
| | | Egg white | 2.57 | | | | |
| | | Soy Protein | | 2.57 | | | |
| | Masking | Sugarcane extract | 1.40 | 1.40 | | | |
| | components | Sucralose | | | 1.40 | | |
| | | Thaumatin | | | | 1.40 | |
| | | Erythritol | | | | | 1.40 |
| | Auxiliary raw | Tapioca starch | 3.00 | 3.00 | 3.00 | 3.00 | 3.00 |
| | materials | Xanthan gum | 0.35 | 0.35 | 0.35 | 0.35 | 0.35 |
| | | Fragrances | 0.65 | 0.65 | 0.65 | 0.65 | 0.65 |
| | | Coloring | 0.30 | 0.30 | 0.30 | 0.30 | 0.30 |

| | | | | | |
|---|---|---|---|---|---|
| total | 100.00 | 100.00 | 100.00 | 100.00 | 100.00 |
| Carbohydrate content (wt %) | 88.43 | 88.43 | 88.43 | 88.43 | 88.43 |
| Acid component content (wt %) | 1.65 | 1.65 | 1.65 | 1.65 | 1.65 |
| Carbonate component content (wt %) | 1.65 | 1.65 | 1.65 | 1.65 | 1.65 |
| Foam stabilizer content (wt %) | 2.57 | 2.57 | 2.57 | 2.57 | 2.57 |
| Mashing component content (wt %) | 1.40 | 3.40 | 1.40 | 1.40 | 1.40 |

| Foamy food product *1 | Powder composition: water | 5.30:1.50 | 5.30:1.50 | 5.30:1.50 | 5.30:1.50 | 5.30:1.50 |
|---|---|---|---|---|---|---|
| | (weight ratio) | | | | | |
| | Specific gravity (g/cc) *2 | 0.37 | 0.38 | 0.41 | 0.42 | 0.41 |
| Masking evaluation *3 | A: Immediately after taking | 0.33 | 0.33 | 0.50 | 1.17 | 1.00 |
| | B: During chewing | 1.33 | 1.00 | 1.17 | 1.83 | 2.00 |
| | (between A and C) | | | | | |
| | C: After swallowing, | 2.00 | 2.33 | 2.17 | 2.50 | 3.00 |
| | Average of A to C | 1.22 | 1.22 | 1.28 | 1.78 | 2.00 |
| | Final evaluation | Excellant | Excellent | Excellent | Good | Good |

TABLE 3
(Parts by weight)

| | | | Example 16 | Example 17 | Example 18 | Example 19 | Example 20 |
|---|---|---|---|---|---|---|---|
| Powder | Carbohydrates | sugar | 53.66 | 71.54 | 71.54 | 71.54 | 71.54 |
| Composition | | Glucose | 2.40 | 3.20 | 3.20 | 3.20 | 3.20 |
| | | Dextrin | 10.27 | 13.69 | 13.69 | 13.69 | 13.69 |
| | Acid component | Citric acid | 1.65 | 0.83 | 1.65 | 1.65 | 2.64 |
| | Carbonate component | Sodium bicarbonate | 1.65 | 0.83 | 1.65 | 1.65 | 0.66 |
| | Foam Stabilizers | Quillaja saponin | 0.43 | 0.43 | 0.22 | 0.43 | 0.43 |
| | | Starch Octenyl Succinate | 2.14 | 2.14 | 1.07 | 2.14 | 2.14 |
| | | Sodium | | | | | |
| | Masking component | Sugarcane extract | 1.40 | 1.40 | 1.40 | 2.80 | 1.40 |
| | Auxiliary raw materials | Tapioca starch | 3.00 | 3.00 | 3.00 | 3.00 | 3.00 |
| | | Xanthan gum | 0.35 | 0.35 | 0.35 | 0.35 | 0.35 |
| | | Fragrances | 0.65 | 0.65 | 0.65 | 0.65 | 0.65 |
| | | Coloring | 0.30 | 0.30 | 0.30 | 0.30 | 0.30 |

| | | | | | |
|---|---|---|---|---|---|
| total | 77.89 | 98.35 | 98.72 | 101.40 | 100.00 |
| Carbohydrate content (wt %) | 85.15 | 89.91 | 89.58 | 87.21 | 88.43 |
| Acid component content (wt %) | 2.12 | 0.84 | 1.67 | 1.63 | 2.64 |
| Carbonate component content (wt %) | 2.12 | 0.84 | 1.67 | 1.63 | 0.66 |
| Foam stabilizer content (wt %) | 3.30 | 2.61 | 1.30 | 2.53 | 2.57 |
| Masking ingredient content (wt %) | 1.80 | 1.42 | 1.42 | 2.76 | 1.40 |

| Foamy food product *1 | Powder composition:water | 4.13:1.50 | 5.21:1.50 | 5.23:1.50 | 5.37:1.50 | 5.30:1.50 |
|---|---|---|---|---|---|---|
| | (weight ratio) | | | | | |
| | Specific gravity (g/co) *2 | 0.33 | 0.53 | 0.43 | 0.39 | 0.67 |
| Masking evaluation *3 | A: Immediately after taking | 0.50 | 0.67 | 0.50 | 0.67 | 0.83 |
| | B: During chewing | 1.33 | 1.67 | 1.33 | 1.67 | 1.83 |
| | (between A and C) | | | | | |
| | C: After swallowing | 3.33 | 2.67 | 2.83 | 2.67 | 3.33 |
| | Average of A to C | 1.72 | 1.67 | 1.56 | 1.67 | 2.00 |
| | Final evaluation | Good | Good | Good | Good | Good |

(Parts by weight)

| | | | Example 21 | Example 22 | Example 23 | Example 24 |
|---|---|---|---|---|---|---|
| Powder | Carbohydrates | sugar | 71.54 | 71.54 | 71.54 | 71.54 |
| Composition | | Glucose | 3.20 | 3.20 | 3.20 | 3.20 |
| | | Dextrin | $3.69 | 13.69 | 13.69 | 13.69 |
| | Acid component | Citric acid | 1.98 | 1.32 | 0.66 | 0.66 |
| | Carbonate component | Sodium bicarbonate | 1.32 | 1.98 | 2.64 | 2.64 |
| | Foam Stabilizers | Quillaja saponin | 0.43 | 0.43 | 0.43 | 0.43 |
| | | Starch Octenyl Succinate | 2.14 | 2.14 | 2.14 | 2.14 |
| | | Sodium | | | | |
| | Masking component | Sugarcane extract | 1.40 | 1.40 | 1.40 | 0.70 |
| | Auxiliary raw materials | Tapioca starch | 3.00 | 3.00 | 3.00 | 3.00 |
| | | Xanthan gum | 0.35 | 0.35 | 0.35 | 0.35 |
| | | Fragrances | 0.65 | 0.65 | 0.65 | 0.65 |
| | | Coloring | 0.30 | 0.30 | 0.30 | 0.30 |

| | | | | |
|---|---|---|---|---|
| total | 100.00 | 100.00 | 100.00 | 99.30 |
| Carbohydrate content (wt %) | 88.43 | 88.43 | 88.43 | 89.05 |
| Acid component content (wt %) | 1.98 | 1.32 | 0.86 | 0.66 |
| Carbonate component content (wt %) | 1.32 | 1.98 | 2.64 | 2.66 |
| Foam stabilizer content (wt %) | 2.57 | 2.57 | 2.57 | 2.59 |
| Masking ingredient content (wt %) | 1.40 | 1.40 | 1.40 | 0.70 |

| Foamy food product *1 | Powder composition:water | 5.30:1.50 | 5.30:1.50 | 5.30:1.50 | 5.26:1.50 |
|---|---|---|---|---|---|
| | (weight ratio) | | | | |
| | Specific gravity (g/co) *2 | 0.44 | 0.38 | 0.53 | 0.39 |
| Masking evaluation *3 | A: Immediately after taking | 0.83 | 0.50 | 0.83 | 0.50 |

| | | | | |
|---|---|---|---|---|
| B: During chewing | 1.67 | 1.33 | 1.83 | 1.17 |
| (between A and C) | | | | |
| C: After swallowing | 3.17 | 2.83 | 3.17 | 2.67 |
| Average of A to C | 1.89 | 1.56 | 1.94 | 1.44 |
| Final evaluation | Good | Good | Good | Excellent |

As shown in Tables 2 and 3, excellent masking effects were observed in all the examples.

### [Confirmation Test of Masking Effect Due to Different Forms]

The bitterness masking effect due to the difference in food form was evaluated. Specifically, the masking effect was compared between the foamy food product prepared from the powder composition of Example 1 and Example 2 above and a known medication-assisting food.

The specific method is as follows: The bittering agent used was the same as that used in Example 1 above.

### <Preparation of Foamy Food Product and Foamy Food Product Containing Bittering Agent>

For Examples 1 and 2, foamy food products and foamy food products containing a bittering agent were prepared in the same manner as Examples 1 and 2 in Table 1 (*1 in Table 4).

### <Preparation of Medication-Assisting Foods and Medication-Assisting Foods Containing Bittering Agents> (*2 in Table 4)

In Reference Example 1, 0.5 g of a bittering agent was dissolved in 1.5 cc of water by stirring to prepare a liquid medication-assisting food containing a bittering agent.

In Reference Example 2, 0.5 g of a bittering agent was enveloped in 25 g of “Okusuri-Nometane (registered trademark) Strawberry Flavor (product name)” (manufactured by Ryukakusan Co., Ltd.) to prepare a jelly-like medication-assisting food containing the bittering agent.

For Reference Example 3, 0.5 g of a bittering agent was enveloped in 18 g of “Okusuri-Nometane (registered trademark) Chocolate Flavor (product name)” (manufactured by Ryukakusan Co., Ltd.) to prepare a jelly-like medication-assisting food containing the bittering agent.

### <Masking Evaluation>

The obtained foamy food product or medication-assisting food was mixed with a bittering agent to prepare a bittering agent-containing food, which was then taken and evaluated (*3 in Table 4). The evaluation was performed by six expert panelists. The specific method is as follows.
0.5 g of the bittering agent was placed in the mouth in the same way as a normal powdered drug and washed down with water. The maximum bitterness felt when this was measured was the upper limit (5 points), and the lower limit (0 points) was the complete lack of bitterness felt.
Relative evaluation was done on a 6-point scale ranging from 5 points (maximum bitterness) to 0 points (no bitterness felt at all).
Evaluation was conducted at three points: A: immediately after taking the tablet (the first taste felt when put in the mouth), B: during chewing (a taste between A and C), and C: after swallowing (the taste that lingers after swallowing).
Evaluations A to C are the average of six expert panelists.
The final evaluation was determined as follows based on the average of A to C.
Excellent: 0 points or more, 1.5 points or less
Good: more than 1.5 points, 2 points or less
Bad: more than 2 points, 5 points or less

The results are shown in Table 4.

TABLE 4
(Parts by weight)

| | | | | Reference | Reference | Reference |
|---|---|---|---|---|---|---|
| | | Example 1 | Example 2 | Example 1 | Example 2 | Example 3 |
| Foamy food product (*1) or | forn | Foamy | Foamy | Liquid | Jelly | Jelly |
| Medication assisting food (*2) | Specific Gravity (g/cc) | 0.39 | 0.43 | 1.00 | 1.02 | 1.05 |
| | pH | 5.2 | 6.2 | 7.0 | 3.7 | 7.0 |
| Masking evaluation X3 | A: Immediately after taking | 0.50 | 0.50 | 3.83 | 2.17 | 2.83 |
| | B: During chewing (between A and C) | 1.33 | 1.00 | 4.17 | 3.50 | 3.67 |
| | C: After swallowing | 2.83 | 2.37 | 4.50 | 4.00 | 4.00 |
| | Average of A to C | 1.50 | 1.22 | 4.17 | 3.22 | 3.50 |
| | Final evaluation | Good | Excellent | Bad | Bad | Bad |

As a result of the evaluation, Reference Examples 2 and 3 were slightly masked compared to Reference Example 1, but the bitterness was felt and hardly suppressed. On the other hand, it was confirmed that Examples 1 and 2 obtained a masking effect far exceeding that of Reference Examples 2 and 3. In particular, there was an overwhelming difference immediately after taking the drug, and Examples 1 and 2 hardly felt bitterness.

### [Dissolution Test]

A drug was mixed into the foamy food product of the present invention to confirm the dissolution of the drug. Specifically, the foamy food product prepared from the powder composition of Example 2 in the above masking effect confirmation test was mixed with the four drugs listed in Table 5 below, and a dissolution test was performed according to the method of the Japanese Pharmacopoeia. This dissolution test is performed to determine whether the oral preparation complies with the dissolution test standard.

The forms of the four drugs shown in Table 5 are as follows:
Acetaminophen: fine granules
Prednisolone: Tablets
Furosemide: fine granules
Metronidazole: tablets

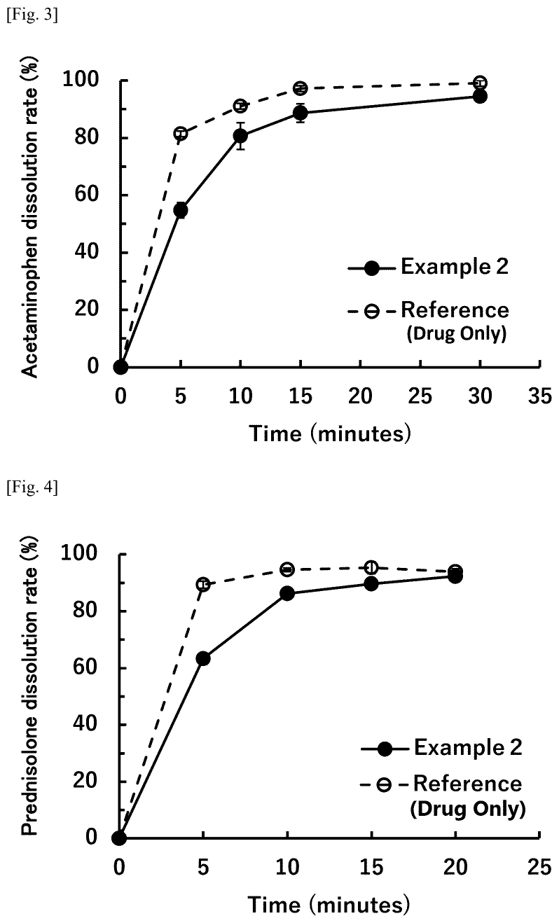

As a control, the drug was not included in the foamy food product and the dissolution test was performed on the drug alone. The dissolution standards of each drug are also shown in Table 5. The results of the dissolution test are shown in FIG. 3 to FIG. 6.

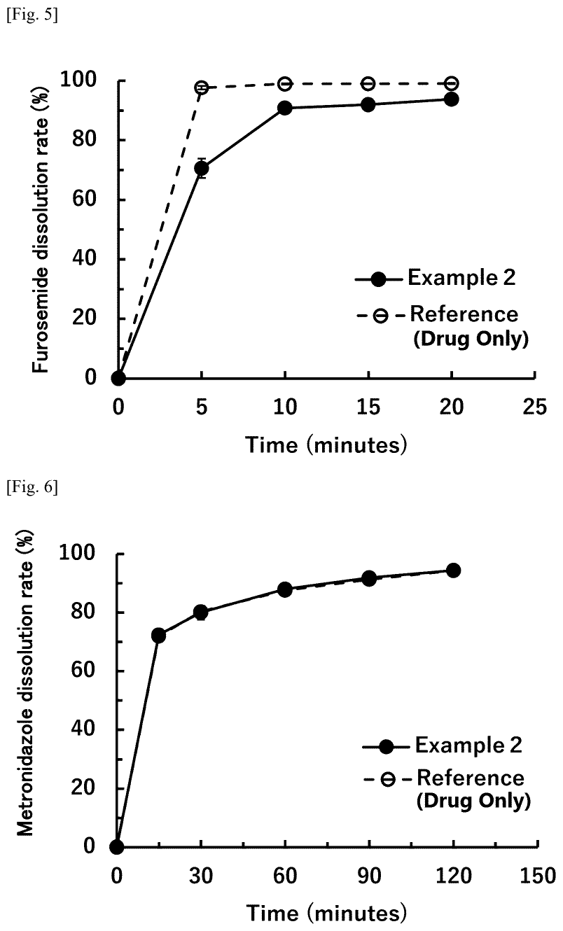

TABLE 5

| | Drugs (active pharmaceutical | |
|---|---|---|
| | ingredients) | Dissolution Standards |
| FIG. 3 | Acetaminophen | 30 minutes 80% or more |
| FIG. 4 | Prednisolone | 20 minutes 70% or more |
| FIG. 5 | Furosemide | 15 minutes 85% or more |
| FIG. 6 | Metronidazole | 90 minutes 75% or more |

As shown in FIG. 3 to FIG. 6, the foamy food product of Example 2 including a drug met the dissolution standards for all four types of drugs tested. In other words, it was shown that the foamy food product of the powder composition of the present invention does not affect the dissolution of the drug.

### [Post-Meal Questionnaire Survey]

### Example 25

After preparing the powder composition of Example 2, it was filled into an aluminum bag and then sealed. Furthermore, the container 100 of FIG. 2 and a plastic spoon were prepared, and these were sealed together in one package to prepare a combined food for masking the bitterness of drug (Example 25).

Example 25 and the bittering agent described below were provided to 30 boys and girls aged 1 to 9 years old, and a foamy food product and a foamy food product containing a bittering agent were prepared and eaten in the same manner as in Example 2 in Table 1, and a questionnaire survey was conducted. Each of the 30 boys and girls aged 1 to 9 years old was accompanied by one parent to prepare the food, and the 30 boys and girls aged 1 to 9 years old consumed the food. Next, “Okusuri-Nometane (registered trademark) Chocolate flavor (product name)” (manufactured by Ryukakusan Co., Ltd.) and the bittering agent described below were provided, and a jelly-like medication-assisting food containing a bittering agent was prepared and eaten in the same manner as in Reference Example 3 in Table 4, and a questionnaire survey was conducted in the same manner. Bittering agent: 4% DC Bitter #20023 and 96% corn starch were mixed and extrusion granulation was carried out to prepare a granular bittering agent.

Regarding Example 25, after explaining the specifications as described below, Questionnaire 1 was conducted on the parents.
Powder composition, container and spoon are included.
All you need to do is prepare water at home.
A total of 6 servings is contained.
The tray and spoon can be washed and reused.
All 6 doses are divided into small portions and can be stored for up to 24 months.
The effectiveness of the medication will not be impaired.
Even if you don't have a measuring spoon, you can measure water using the depression line of the tray.

### <Questionnaire 1>

“If there was a medication-assisting food like Example 25, how likely would you be to buy it?”
Really want to buy
Want to buy a little
Neither (I don't know)
Don't really want to buy
Don't want to buy

For Example 25, foamy food product and bitter agent-containing foamy food product were prepared and eaten, and then questionnaires 2 and 3 were conducted for boys and girls aged 1 to 9 years old, and questionnaires 4 and 5 were conducted for their parents. For Reference Example 3, a jelly-like medication-assisting food containing bittering agent was prepared and eaten, and a similar questionnaire was conducted.

### <Questionnaire 2>

“Did you feel bitter taste or unpleasant taste of the drug (bittering agent)?”
Didn't feel taste of drug at all
Didn't feel much taste of drug
Neither
Felt a little taste of drug
Felt much taste of drug

### <Questionnaire 3>

“How easy was it to take the drug (bittering agent)?”
It was easy to eat
It was a little easy to eat
It was a little hard to eat
It was difficult to eat

### <Questionnaire 4>

“How was your child?”
He/She ate it without any complaints
He/She was a little reluctant but ate it anyway
Neither
He/She didn't like it and didn't eat much
He/She hated it and hardly ate anything

### <Questionnaire 5>

“How easy was it to get your child to take the drug (bittering agent)?”
Easy to feed
A little easy to feed
A little difficult to feed
Difficult to feed

Finally, questionnaire 6 was conducted targeting parents.

### <Questionnaire 6>

“Which would you like to buy, Example 25 or Reference Example 3?”
Example 25
a little Example 25
a little Example 3
Reference Example 3

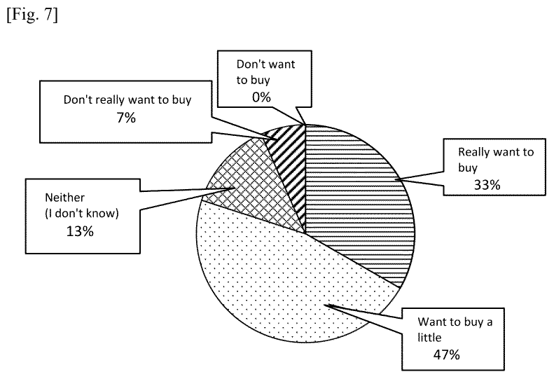

The results of the questionnaire survey, as set forth in Table 6, are shown in FIG. 7 to FIG. 12.

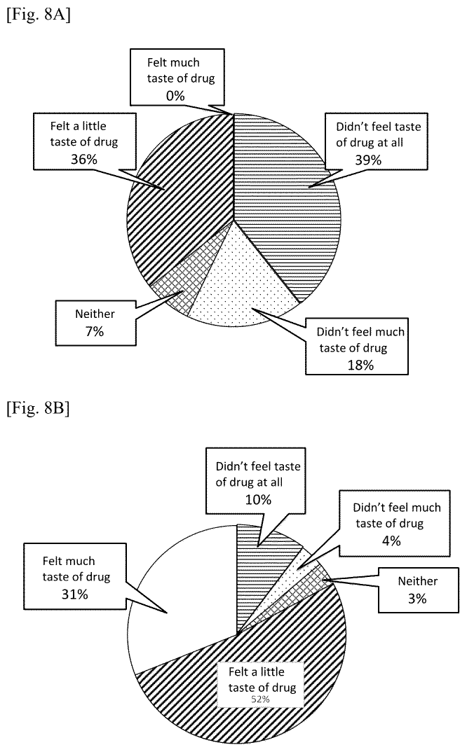

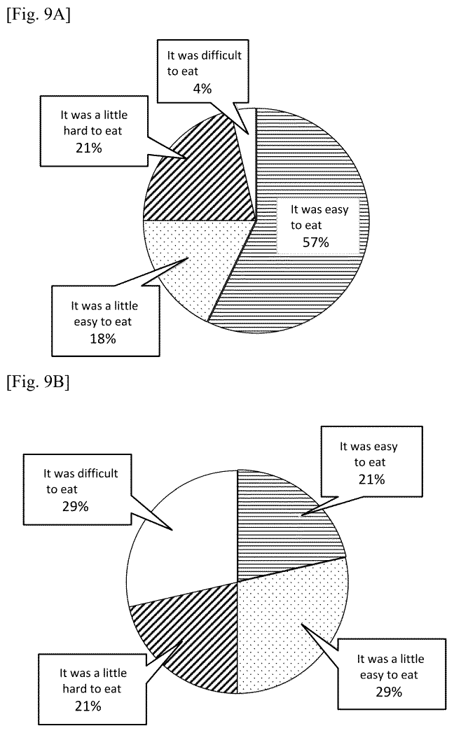

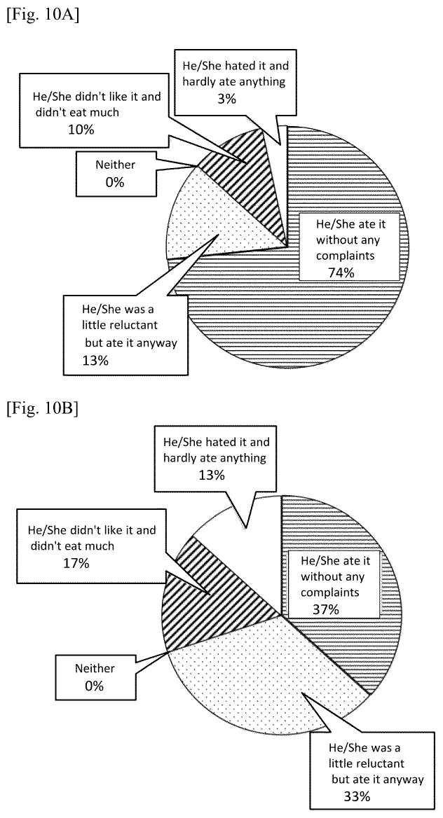

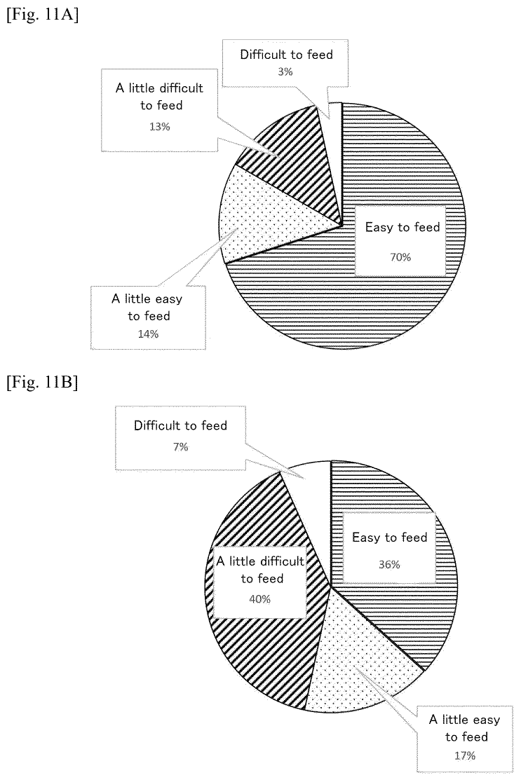

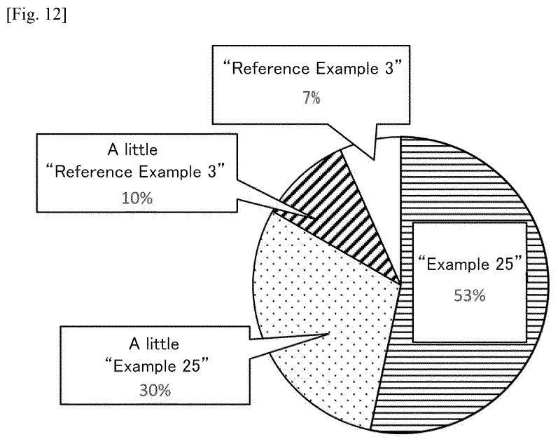

TABLE 6

| | | Referencial |
|---|---|---|
| | Example 25 | Example 3 |
| Questionnaire 1 | FIG. 7 | — |
| Questionnaire 2 | FIG. 8A | FIG. 8B |
| Questionnaire 3 | FIG. 9A | FIG. 9B |
| Questionnaire 4 | FIG. 10A | FIG. 10B |
| Questionnaire 5 | FIG. 11A | FIG. 11B |

| | |
|---|---|
| Questionnaire 6 | FIG. 12 |

The results of questionnaires 2 to 5 showed that Example 25 has a masking effect on the bittering agent, and that the masking effect makes it easy to ingest the bittering agent.

In addition, the results of the questionnaire survey showed that consumers want medication-assisting foods with a masking effect other than the conventional jelly-like medication-assisting foods, and that the product of the present invention has a masking effect superior to that of conventional medication-assisting foods.

It should be considered that the embodiments disclosed this time are illustrative in all respects and not restrictive. The scope of the present invention is indicated by the scope of the claims rather than the above description, and is intended to include all modifications within the scope and meaning equivalent to the scope of the claims.

### REFERENCE SIGNS LIST

10 Drug-including foamy food product
11 Foamy food product
12 Air bubbles
13 Drug
100 Container
110 Main body
111 Raised part
111a Top surface
112 Two-tiered recess
113 First recess
114 Second recess
115 Groove
120 Spoon part
121 Third recess
130 Perforation

1. A powder composition for mixing with water to form a foamy food product, comprising the following components (A) to (D).
(A) a carbohydrate
(B) an acid component
(C) a carbonate component
(D) a foam stabilizer

2. The powder composition according to claim 1, further comprising the following component (E).
(E) a masking component

3. The powder composition according to claim 1, wherein the foamy food product has a specific gravity of 0.83 g/cc or less.

4. The powder composition according to claim 1, wherein the foamy food product is for enveloping a drug and taking it.

5. A combined food for masking bitterness of drug, comprising: a powder composition;
and
(X) a container having a two-tiered recess in the shape of an inverted Kagami-mochi, the powder composition containing the following components (A) to (D);
(A) a carbohydrate
(B) an acid component
(C) a carbonate component
(D) a foam stabilizer.

6. A method for masking bitterness of drug, comprising the steps of:
adding water to and mixing a powder composition comprising a carbohydrate, an acid component, a carbonate component, and a foam stabilizer to prepare a foamy food product, and
mixing the drug into the foamy food product.

7-8. (canceled)

9. The powder composition according to claim 1, containing at least one of egg white, soy protein, Quillaja saponin, and starch sodium octenyl succinate as the foam stabilizer (D).

10. The powder composition according to claim 1, wherein the carbohydrate (A) is a powdered sweetener, and
the carbohydrate (A) is contained in an amount of 85.0% by weight or more of the total weight of the powder composition.

11. The combined food according to claim 5, containing at least one of egg white, soy protein, Quillaja saponin, and starch sodium octenyl succinate as the foam stabilizer (D).

12. The combined food according to claim 5, further comprising the following component (E).
(E) a masking component

13. The combined food according to claim 5, wherein the powder composition becomes a foamy food product when water is added and mixed, and
the foamy food product has a specific gravity of 0.83 g/cc or less.

14. The combined food according to claim 5, wherein the powder composition becomes a foamy food product when water is added and mixed, and
the foamy food product is for enveloping a drug and taking it.

15. The combined food according to claim 5, wherein the carbohydrate (A) is a powdered sweetener, and
the carbohydrate (A) is contained in an amount of 85.0% by weight or more of the total weight of the powder composition.

16. The method for masking bitterness of drug according to claim 6, containing at least one of egg white, soy protein, Quillaja saponin, and starch sodium octenyl succinate as the foam stabilizer.

17. The method for masking bitterness of drug according to claim 6, wherein the powder composition further comprises a masking component.

18. The method for masking bitterness of drug according to claim 6, wherein the specific gravity of the foamy food product is 0.83 g/cc or less.

19. The method for masking bitterness of drug according to claim 6, wherein the foamy food product is for enveloping a drug and taking it.

20. The method for masking bitterness of drug according to claim 6, wherein the carbohydrate is a powdered sweetener, and
the carbohydrate is contained in an amount of 85.0% by weight or more of the total weight of the powder composition.
